(19) United States
(12) Patent Application Publication
(10) Pub. No.: US 2020/0138998 A1
(43) Pub. Date: May 7, 2020

(54) Room Deodorizer

(71) Applicant: Carleston O. Brown, Euclid, OH (US)

(72) Inventor: Carleston O. Brown, Euclid, OH (US)

(21) Appl. No.: 16/674,998

(22) Filed: Nov. 5, 2019

Related U.S. Application Data
(60) Provisional application No. 62/755,823, filed on Nov. 5, 2018.

(51) Int. Cl. A61L 9/12

(52) U.S. Cl. CPC A61L 9/122; A61L 2209/14; A61L 2209/111

(57) ABSTRACT

The present invention is intended to provide users with a quietly operating, fragrance emitting apparatus that can be placed in houses or any spaces that require a pleasant smell. The present invention is compactly sized such that a homeowner can conveniently place the apparatus in smaller spaces, such as bathrooms and/or laundry rooms. In order to accomplish its purpose, the present invention comprises a blower device that enables to spread the fragrance in the room where the apparatus is placed. Further, the blower device may be actuated manually with the push of a button, or automatically, wherein the blower device will turn on with the help of an environmental sensor attached within the apparatus. Furthermore, the present invention comprises a lighting device that will turn on automatically when the environmental sensor is turned on. Thus, the present invention provides a compact and quite apparatus, that enables users to have light as well as a pleasant smell, when they enter a room or specific location, where the apparatus is placed.

### FIELD OF THE INVENTION

The present invention relates generally to an apparatus that provides a pleasant smell. Specifically, this present invention relates to an apparatus that can quietly provide a pleasant smell, wherein the apparatus gets actuated either manually or automatically.

### BACKGROUND OF THE INVENTION

A pleasantly fragranced room can often make people fall into a good mood immediately. Conversely, a bad smelling room can affect the mood of a person negatively as well. Homeowners often find that their bathrooms and laundry rooms will start having a strong odor after high traffic use. Current solutions to the strong odor are often inadequate. Some solutions use an apparatus that produces pleasant smells to mask the strong odor. However, the apparatus used in said solutions are often too large to be conveniently placed in a bathroom or a laundry room. Other solutions use mechanical ventilation devices to improve the quality of air. Unfortunately, these mechanical ventilation devices produce loud noises when in use. As a result, there is a strong need for an apparatus that improves the smell in bathrooms and laundry rooms while still being small and quiet. Furthermore, an apparatus that is compact, works quietly, and automatically turns on in the presence of the user, or preference of the user is a rare find in the current market.

The present invention is an apparatus that addresses the previously mentioned issues. It is an aim of the present invention, to provide users with an apparatus that can be conveniently placed in their bathrooms, laundry rooms, stairway, hallway, or in any part of a house, a hospital, a commercial building, or even a vehicle. Accordingly, the present invention comprises a blower device that enables to spread the fragrance in the location where the apparatus is placed, in a quite manner. Further, the blower device may be actuated manually with the push of a button, or automatically, wherein the blower device will turn on with the help of an environmental sensor attached within the apparatus. Furthermore, the present invention comprises a lighting device that will turn on automatically when the blower device is turned on. Thus, the present invention provides a compact and quite apparatus, that enables users to have light as well as a pleasant smell, when they enter a room or specific location, where the apparatus is placed.

### BRIEF DESCRIPTION OF THE DRAWINGS

FIG. 1 is a block diagram of the present invention, wherein arrowed flowlines represent electrical/electronic connections between components, and dashed flow lines indicate the components being communicably coupled.

FIG. 2 is a top-front-left perspective view of the present invention.

FIG. 3 is a bottom-rear-left perspective view of the present invention.

FIG. 4 is a top plan view of the present invention.

FIG. 5 is a sectional view of FIG. 4 along AA′.

FIG. 6 is an exploded, top-front-left perspective view of the present invention.

FIG. 7 is a top-front-left perspective view of an alternate embodiment of the present invention.

FIG. 8 is a bottom-rear-right perspective view of an alternate embodiment of the present invention.

FIG. 9 is a top plan view of an alternate embodiment of the present invention.

FIG. 10 is an exploded, bottom-front-left perspective view of an alternate embodiment of the present invention.

FIG. 11 is an exploded perspective view of FIG. 7.

FIG. 12 is an exploded bottom front left perspective view of an alternate embodiment of the present invention, wherein the embodiment in FIG. 7 has a fragrance tray attached to the lid.

FIG. 13 is a bottom-rear-right perspective view of an alternate embodiment of the present invention, wherein the embodiment in FIG. 7 has an environmental sensor attached to a front surface of the device.

### DETAIL DESCRIPTIONS OF THE INVENTION

All illustrations of the drawings are for the purpose of describing selected versions of the present invention and are not intended to limit the scope of the present invention.

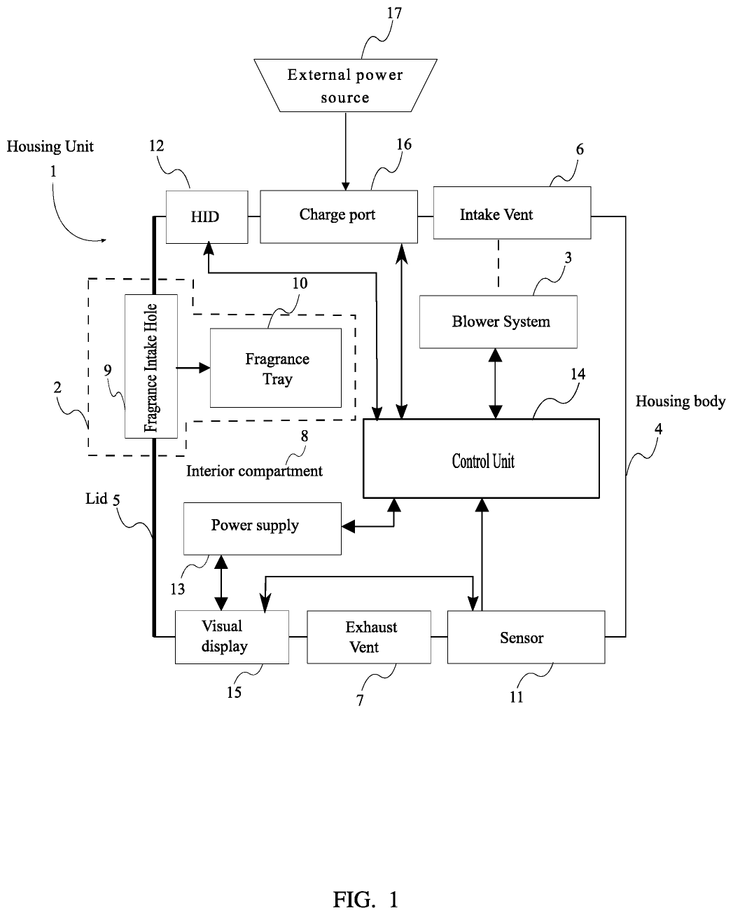

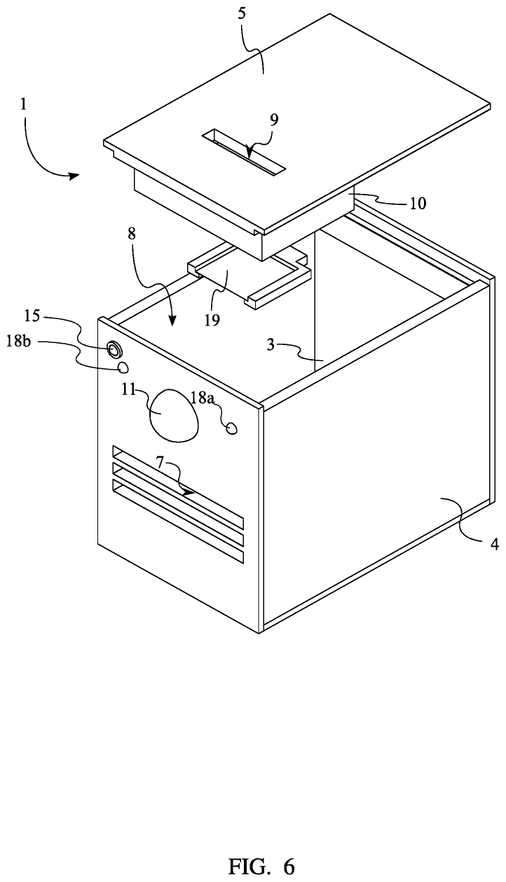

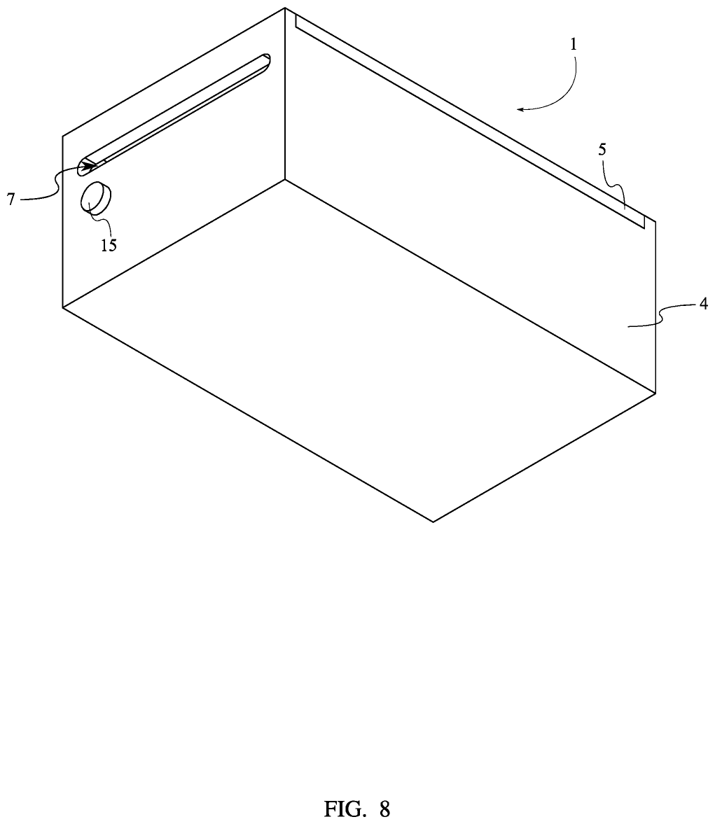

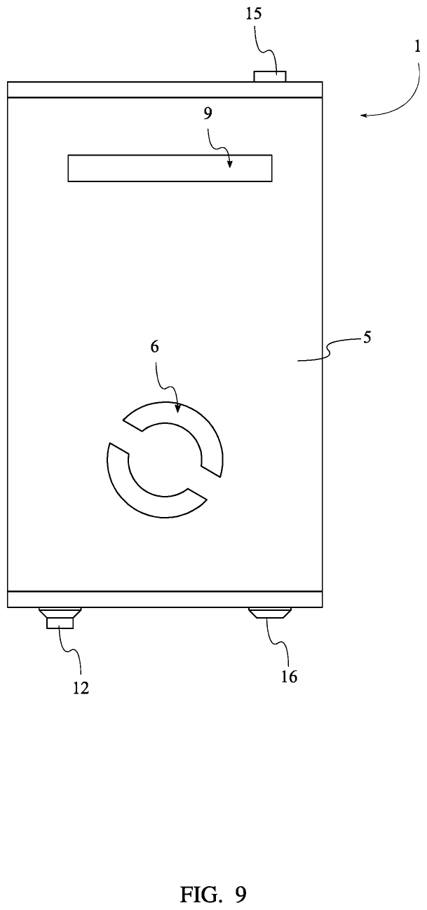

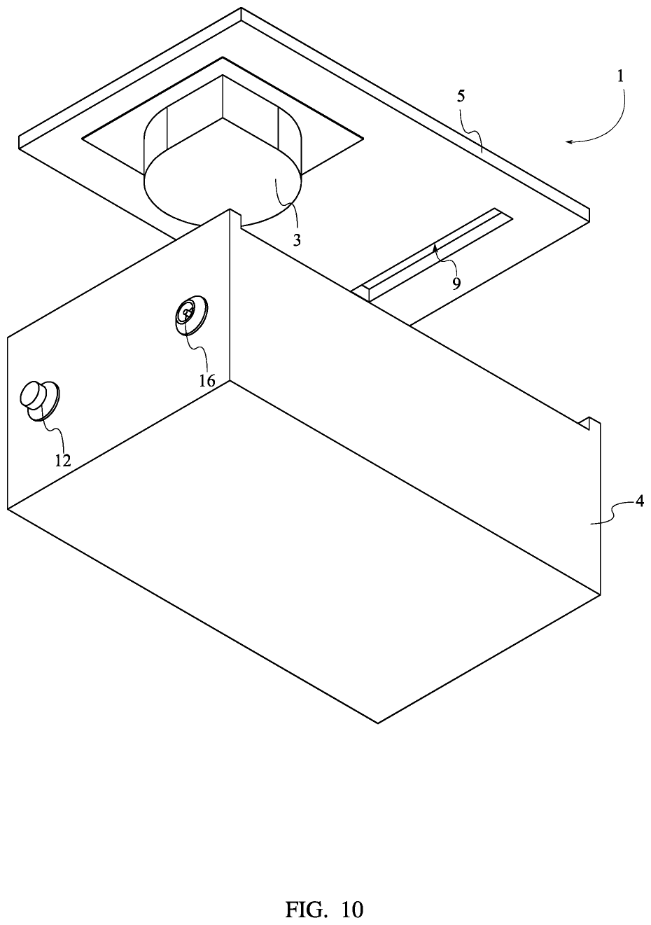

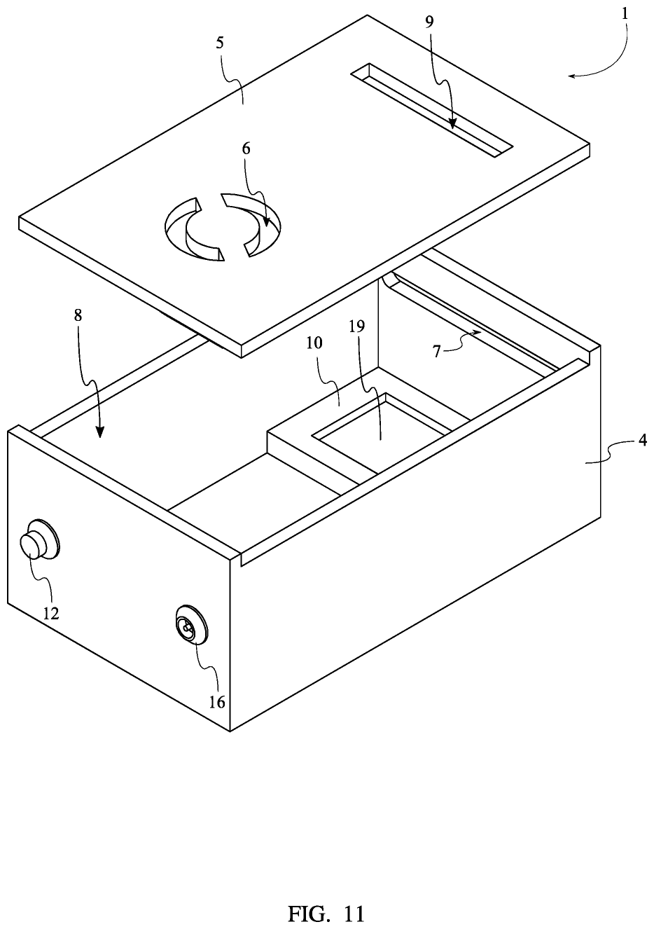

In reference to FIG. 1 through FIG. 13, the present invention is a room deodorizer. It is an aim of the room deodorizer to provide users with a compact and quite fragrance emitting apparatus that can be placed in any location where a pleasant smell is required. To accomplish this, the room deodorizer comprises a blower device that enables to spread the fragrance in the location where the apparatus is placed. Furthermore, the blower device may be actuated manually with the push of a button, or automatically, wherein the blower device will turn on with the help of an environmental sensor attached within the apparatus. Thus, the room deodorizer is a quietly running, compact apparatus, that enables users to have a pleasant-smelling bathroom, laundry room, bedroom, commercial space, office space, hallway etc.

In reference to FIG. 1, the present invention is a room deodorizer that emits fragrance in any space in a quite manner. In order to accomplish this, the present invention comprises a housing unit 1 which provides a structural enclosure for the other components of the present invention, a fragrance attachment 2 which provides the source of a pleasant smell, and a blower system 3, which helps emit the fragrance from the apparatus into the room. In the preferred embodiment of the present invention, the housing unit 1 is in the shape of a rectangular prism, but in other embodiments the housing unit can be of any shape. The housing unit has a volume that is small enough to be conveniently placed in a smaller space such as a bathroom or a laundry room.

In reference to FIG. 1-FIG. 13, the housing unit 1 comprises a housing body 4, a lid 5, an intake vent 6, and an exhaust vent 7. The lid 5 is a cover mounted over an interior compartment 8 of the housing body 4. Further, the intake vent 6 and the exhaust vent 7 traverse through the housing body 4 into the interior compartment 8. Continuing in reference to FIG. 1-FIG. 12, the blower system 3 is mounted within the interior compartment 8 adjacent the intake vent 6, and the intake vent 6 allows for air to come into the housing unit. Similarly, the exhaust vent 7 allows for air that is inside the housing unit 1 to exit the housing unit 1.

In order to fulfill the intended purpose of the present invention, the room deodorizer needs components that can hold and spread fragrances. In reference to FIG. 1 through FIG. 6, the fragrance attachment 2 comprises a fragrance tray 10, a fragrance intake hole 9 and a fragrance receptacle 10a. The fragrance tray 10 is mounted adjacent the lid 5, and the fragrance receptacle 10a traverses into the fragrance tray 10. The fragrance intake hole 9 normally traverses through the lid 5, and the fragrance receptacle 10a is concentrically aligned to the fragrance intake hole 9. According the present invention, the blower system 3 is positioned adjacent to the intake vent 6 and creates an airflow that starts from the intake vent 6 and exits the exhaust vent 7. In the preferred embodiment of the present invention, the blower system 3 is a fan that is located inside the housing body 4. Continuing with the preferred embodiment of the present invention, the fan draws air into the intake vent 6 and pushes it out the exhaust vent 7. The blower system 3 is attached to the housing unit 1 by first inserting the blower system 3 into a socket on the housing unit 1. Then one of more fasteners or connectors (3a, 3b, 3c, 3d) engage connector sockets on the housing unit 1 to secure the blower system 3 in place.

In reference to FIG. 1, the room deodorizer further comprises, at least one environmental sensor 11, a human interface device (HID) 12, a power supply device 13, and a control unit 14. Preferably, the environmental sensor 11 and the HID 12 is laterally mounted onto housing unit 1, and the power supply device 13 and the control unit 14 are positioned within the interior compartment 8. According to the present invention, the environmental sensor 11 determines if a condition has been met before activating the blower system 3. In the preferred embodiment of the present invention, the condition that needs to be met to activate the environmental sensor 11 is the presence of motion in front of the environmental sensor 11. In other words, the environmental sensor 11 is a motion sensor. However, any other forms of environmental sensors such as, audible sensor, light sensor, or any other automatic activation systems that are known to one of ordinary skill in the art may be employed, as long as it doesn't hinder the intended purpose of the present invention.

Continuing with the preferred embodiment of the present invention, HID 12 is a switch that is electrically connected between the power supply device 13 and the control unit 14, and enables a user to manually turn the room deodorizer on or off. The power supply device 13 provides power to the room deodorizer when no external power is provided. In the preferred embodiment of the present invention, the power supply device 13 is a rechargeable battery that can be charged by an external power source. Furthermore, the control unit 14 is electronically connected to the blower system 3, the environmental sensor 11, the HID 12, and the power supply device 13.

In reference to FIG. 1, the room deodorizer further comprises a visual output device 15, and a charge port 16. According to the present invention, the visual output device 15 is used to generate a visual representation of the electrical state of the power supply device 13 of the present invention. In other words, the visual output device 15 emits a signal when the battery of the power supply device 13 has a full charge. In the preferred embodiment of the present invention, the visual output device 15 is a light that lights up when the battery of the power supply device 13 has a full charge. Further, the visual output device 15 also turns on when the environmental sensor 11 senses a required condition. Thus, during nighttime or in places where there is no light, the room deodorizer also acts as a night light/guiding light.

Continuing with the preferred embodiment of the present invention, the charge port 16 connects an external power source 17 to the housing unit 1 to power the room deodorizer. In the preferred embodiment of the present invention, charge port 16 comprises a power socket that can be connected to a power outlet with a plug. Further, the visual output device 15 and the charge port 16 are laterally mounted onto the housing unit 1, wherein the charge port 16 is positioned offset from the visual output device 15 across the housing body 4. Furthermore, the visual output device 15 and the charge port 16 are electronically connected to the control circuit.

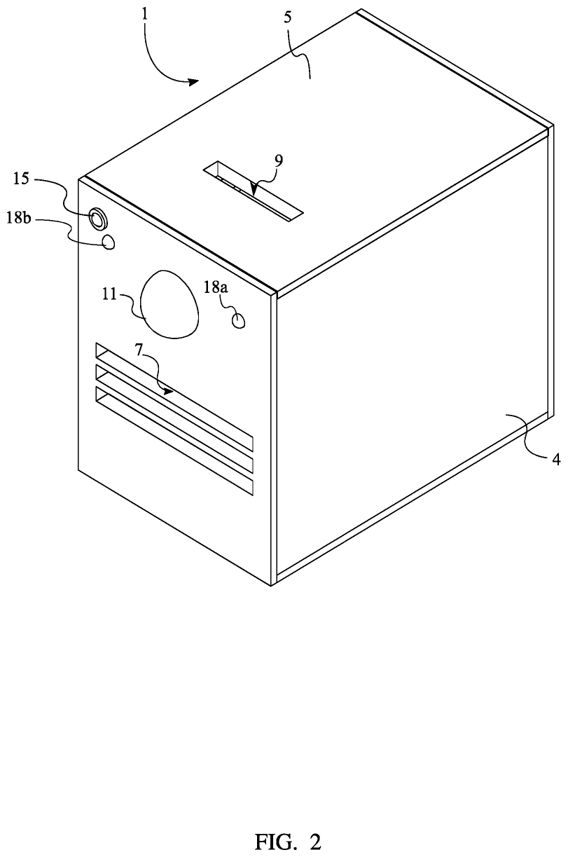

In the preferred embodiment of the present invention, the housing unit 1 is modular and can be assembled without soldering. In reference to FIG. 2-FIG. 6, the housing unit 1 is a rectangular prism and comprises different plates or pieces, that can slide into pre-positioned slots to form the housing unit 1. However, any other fastening methods such as screws, adhesives, magnetic fastening, etc. may be employed to form the housing unit 1.

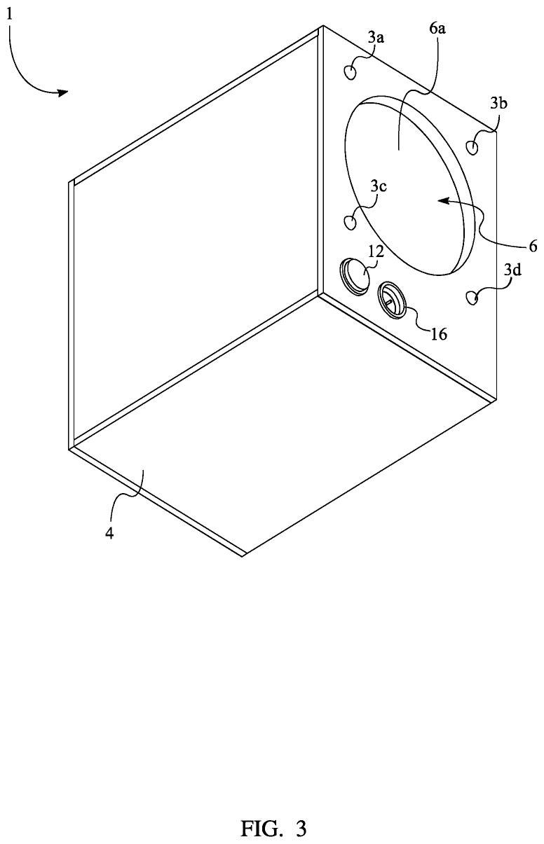

In reference to FIG. 2 and FIG. 3, the present invention comprises a first filter 6a, and a second filter 7a, wherein the first filter 6a is mounted within the interior compartment 8 adjacent the intake vent 6, and wherein the intake vent 6 is in fluid communication with the interior compartment 8 through the first filter 6a. Further, the second filter 7a is mounted within the interior compartment 8 adjacent the exhaust vent 7, wherein the exhaust vent 7 is in fluid communication with the interior compartment 8 through the second filter 7a. Furthermore, according to the preferred embodiment, the exhaust vent 7 comprises a plurality of horizontal openings as seen in FIG. 2, and the intake vent 6 comprises a round opening as seen in FIG. 3. However, the exhaust vent 7 and the intake vent 6 may be positioned at any location along the housing unit 1, and comprise any shapes, as long as the intended purpose of the present invention is not altered.

In reference to FIG. 2, a plurality of connectors 18a, 18b, engages with connector sockets on the housing unit 1, to secure the environmental sensor 11 in place. According to the present invention, an adjustment device may be included, which allows the user to adjust the time the blower system 3 is activated for after the sensor determines a condition has been met. In the preferred embodiment of the present invention, the adjustment device is a screw on the environmental sensor 11, that can be turned to adjust the time the blower system 3 is activated for after the environmental sensor 11 determines a condition has been met.

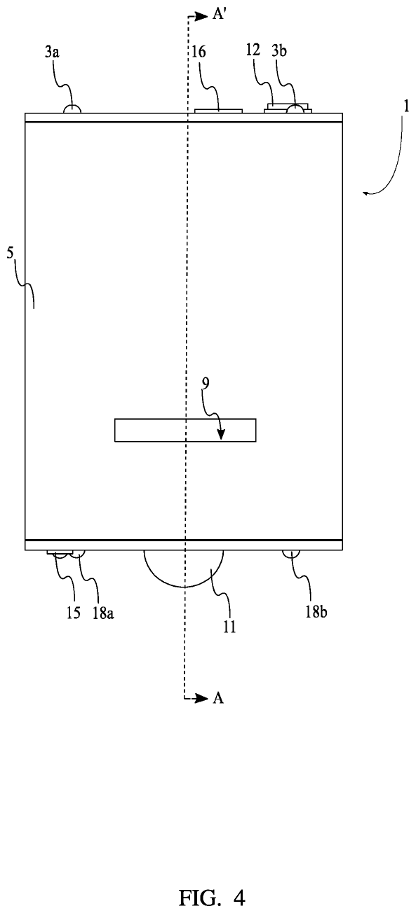

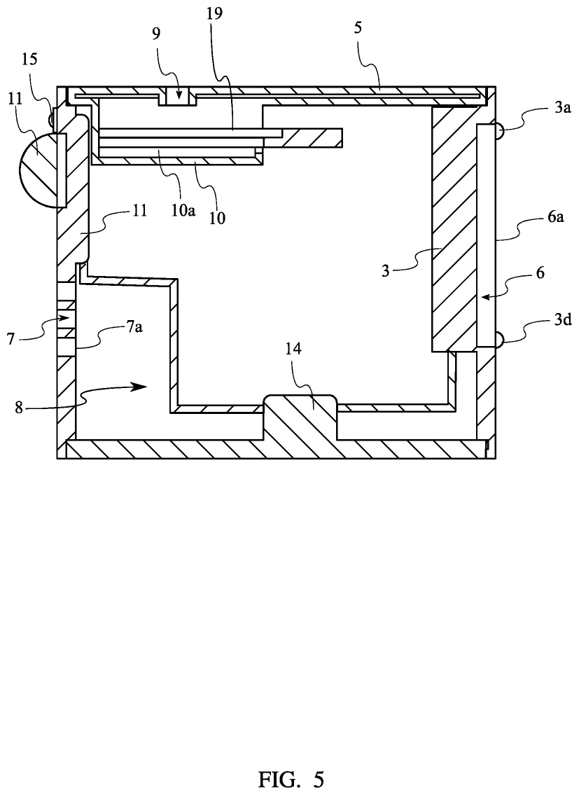

As seen in FIG. 4, the fragrance intake hole 9 is a horizontal slit that traverses through the lid 5. The fragrance intake hole 9 allows the user to put in drops of fragrance oils to the fragrance receptacle 10a, that is disposed within the interior compartment 8, concentrically aligned to the fragrance intake hole 9. The fragrance intake hole 9 can comprise any shape and/or size as long as it doesn't hinder the intended purpose of the present invention. In reference to FIG. 4-FIG. 5, the fragrance attachment comprises the fragrance tray 10, which is securely fastened onto a lower surface of the lid 5, within the interior compartment 8. The fragrance tray 10 may be fastened using any fastening methods that are known to one of ordinary skill in the art, however, preferably, the fragrance tray 10 is attached to adjacent to the lid 5 using an adhesive. Continuing with the preferred embodiment of the present invention, the fragrance receptacle 10a, further comprises a fragrance collecting medium 19 being mounted within the fragrance receptacle 10a. Preferably, the fragrance collecting medium 19 is a fabric that can hold fragrance oils. The fragrance receptacle 10a, the fragrance tray 10, and the fragrance collecting medium 19 can comprise any size, shape, dimensions, material, or type, as long as the intended purpose of the present invention is not altered.

According to a preferred method of operation of the present invention, when the environment al sensor 11 senses a preferred condition and turns on the blower system 3, with the help of the control unit 14, fragrance in the fragrance collecting medium 19 mixes the air from the intake vent 6 with a fragrance. The air that is mixed with the fragrance is pushed out the exhaust vent 7 by the blower system 3, allowing the user to receive a pleasant smell. Accordingly, the fragrance contained in the fabric can be of any fragrance the user wants to use. In the preferred embodiment of the present invention, the fragrance collecting medium 19 is a piece of wool cloth that is treated with an oil-based fragrance of the user's choice. In other embodiments, the fragrance collecting medium 19 can be of any suitable material, that is known to one of ordinary skill in the art.

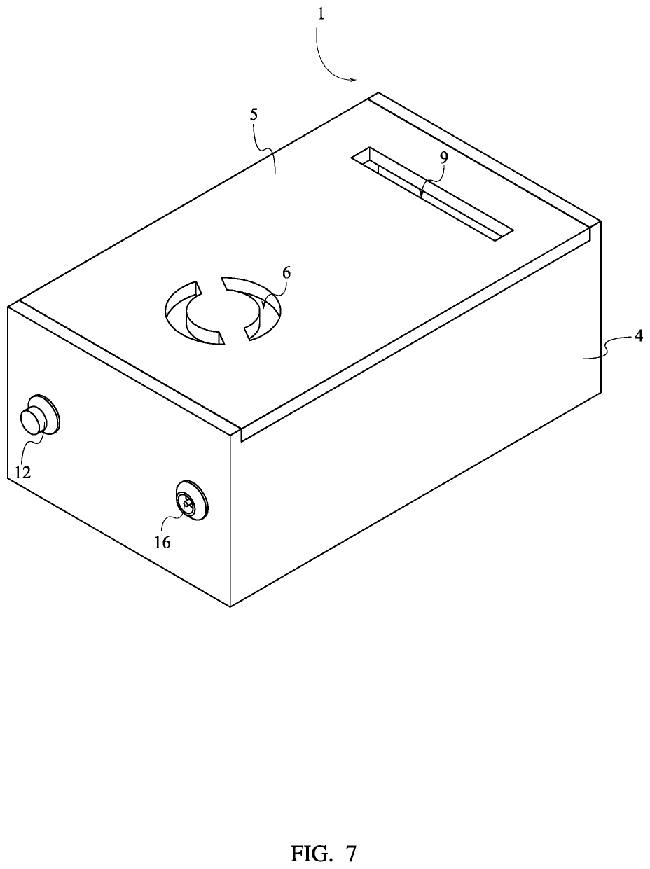

In a first alternative embodiment of the present invention, the room deodorizer is smaller than the apparatus in the preferred embodiment and does not include a fragrance tray 10, nor an environmental sensor 11. In reference to FIG. 7-FIG. 11, the housing unit 1 comprises the fragrance attachment 2, the blower system 3, and the HID 12. Preferably, the housing unit 1 has the shape of a rectangular prism, but any other shape and size may be utilized. According to this embodiment, the fragrance attachment 2 is located inside the housing unit 1 and mixes the air coming from the intake vent 6 with a fragrance. The air that is mixed with the fragrance from the fragrance attachment 2 is blown through the exhaust vent 7 by the blower system 3, allowing the user to receive a pleasant smell. The fragrance attachment 2 comprises the fabric intake hole 9, and the fragrance tray 10 with the fragrance collecting medium 19. The fragrance collecting medium 19 contains a fragrance of the user's choice that will be mixed with the air coming from the intake vent 6. The blower system 3 comprises a fan that is positioned inside the housing unit 1 and adjacent to the intake vent 6. The fan draws air into the intake vent 6 and pushes air out of the exhaust vent 7. The HID 12 allows the user to turn the blower system 3 on or off.

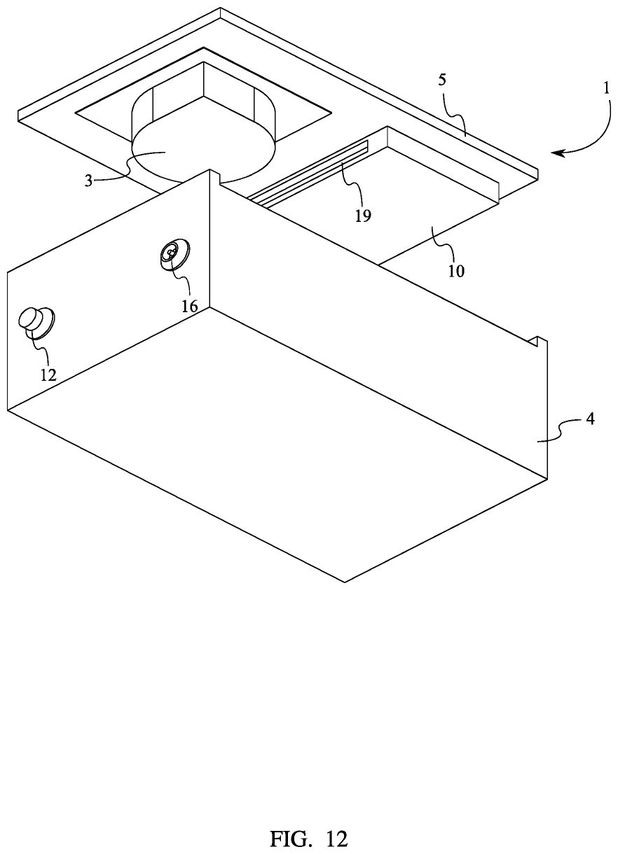

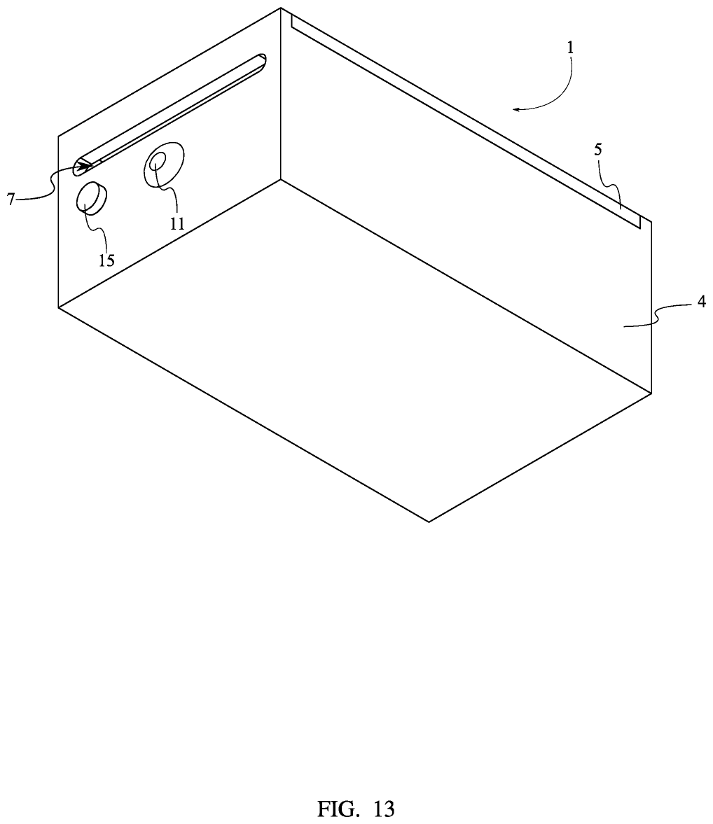

In a second alternate embodiment of the present invention, the room deodorizer is smaller than the preferred embodiment, but contains all the components of the preferred embodiment. In reference to FIG. 12 and FIG. 13, the fragrance tray 10 is attached to the lower surface of the lid, concentrically below the fragrance intake hole 9. The fragrance tray 10 has the fragrance collecting medium 19 disposed within the dimensions of the fragrance tray 10. The user adds drops of fragrance oil through the fragrance intake hole 9, which will be collected by the fragrance collecting medium 19. Continuing with reference to FIG. 13, the room deodorizer also has the environmental sensor 11 mounted within the interior compartment 8 and the housing body 4, wherein the blower system 3 will only operate upon sensing of a condition required by the environmental sensor 11. Accordingly, the visual display device 15 also turns on upon detection of the environmental sensor 11's condition.

Although the invention has been explained in relation to its preferred embodiment, it is to be understood that many other possible modifications and variations can be made without departing from the spirit and scope of the invention.

What is claimed is:

1. A room deodorizer comprising:
a housing unit;
a fragrance attachment;
a blower system;
the housing unit comprising a housing body, a lid, an intake vent, and an exhaust vent;
the lid being mounted over an interior compartment of the housing body;
the intake vent traversing through the housing body into the interior compartment;
the exhaust vent traversing through the housing body into the interior compartment;
the fragrance attachment being mounted adjacent to the lid;
the fragrance attachment being positioned within the interior compartment; and
the blower system being mounted within the interior compartment adjacent the intake vent.

2. The room deodorizer of claim 1, the fragrance attachment comprises:
a fragrance intake hole;
the fragrance attachment comprising a fragrance tray and a fragrance receptacle;
the fragrance intake hole normally traversing through the lid;
the fragrance tray being mounted adjacent to the lid;
the fragrance receptacle traversing into the fragrance tray; and
the fragrance receptacle being concentrically aligned to the fragrance intake hole.

3. The room deodorizer of claim 2, wherein:
the fragrance attachment further comprising a fragrance collecting medium; and
the fragrance collecting medium being mounted within the fragrance receptacle.

4. The room deodorizer of claim 2, wherein the fragrance collecting medium is a fabric that can hold fragrance oils.

5. The room deodorizer of claim 1, further comprising:
at least one environmental sensor;
a human interface device (HID);
a power supply device;
a control circuit;
the at least one environmental sensor being laterally mounted onto the housing unit;
the human interface device (HID) being laterally mounted onto housing unit;
the power supply device being mounted within the interior compartment; and
the control circuit being positioned within the interior compartment.

6. The room deodorizer of claim 5, wherein the control circuit is electronically connected to the blower system, the at least one environmental sensor, the HID, and the power supply device.

7. The room deodorizer of claim 5, wherein the at least one environmental sensor is a motion sensor.

8. The room deodorizer of claim 5, wherein the blower module is a fan

9. The room deodorizer of claim 5, wherein the HID is a switch.

10. The room deodorizer of claim 5, further comprising:
a visual output device;
a charge port;
the visual output device being laterally mounted onto the housing unit;
the charge port being laterally mounted onto the housing unit;
the charge port being positioned offset from the visual output device across the housing body; and
the visual output device and the charge port being electronically connected to the control circuit;

11. The room deodorizer of claim 5, wherein the at least one sensor and/or the HID triggers the operation of the blower system.

12. The room deodorizer of claim 1, further comprising:
a first filter;
a second filter;
the first filter being mounted within the interior compartment adjacent the intake vent;
the intake vent being in fluid communication with the interior compartment through the first filter;
the second filter being mounted within the interior compartment adjacent the exhaust vent; and
the exhaust vent being in fluid communication with the interior compartment through the second filter.

13. A room deodorizer comprising:
a housing unit;
a fragrance attachment;
a blower system;
the housing unit comprising a housing body, a lid, an intake vent, and an exhaust vent;
the lid being mounted over an interior compartment of the housing body;
the intake vent traversing through the housing body into the interior compartment;
the exhaust vent traversing through the housing body into the interior compartment;
the fragrance attachment being positioned within the interior compartment; and
the blower system being mounted within the interior compartment adjacent the intake vent.

14. The room deodorizer of claim 13, wherein:
the fragrance attachment further comprising a fragrance collecting medium and a fragrance receptacle; and
the fragrance collecting medium being mounted within the fragrance receptacle.

15. The room deodorizer of claim 13, further comprising:
a human interface device (HID);
a power supply device;
a control circuit;
the human interface device (HID) being laterally mounted onto housing unit;
the power supply device being mounted within the interior compartment; and
the control circuit being positioned within the interior compartment.

16. The room deodorizer of claim 15, wherein the control circuit is electronically connected to the blower system, the at least one environmental sensor, the HID, and the power supply device.

17. The room deodorizer of claim 15, wherein:
the blower module is a fan; and
the HID is a switch.
